(19) United States
(12) Patent Application Publication
(10) Pub. No.: US 2012/0022160 A1
(43) Pub. Date: Jan. 26, 2012

(54) COMPOSITION FOR EXTERNAL USE ON SKIN

(76) Inventors: Kazunobu Suzuki, Kanagawa (JP); Naoki Tominaga, Kanagawa (JP)

(21) Appl. No.: 13/124,114

(22) PCT Filed: Oct. 14, 2009
(86) PCT No.: PCT/JP2009/005355

§ 371 Date: Jul. 27, 2011

(30) Foreign Application Priority Data
Oct. 15, 2008 (JP) 2008-265965
Oct. 8, 2009 (JP) 2009-234521

(51) Int. Cl. A61K 8/92; A61Q 19/00; A61K 8/73

(52) U.S. Cl. 514/561

(57) ABSTRACT

It is intended to provide a cleansing cosmetic composition that quickly removes makeup from the skin, is easily applicable onto the skin without dripping from the hand or running on the face during application, and can freshen the skin because of being washed off thoroughly. The present invention provides a cleansing cosmetic composition comprising: (1) 1 to 15% by mass of silicone oil, (2) 1 to 15% by mass of volatile hydrocarbon oil, (3) 5 to 20% by mass of dihydric glycol, (4) 5 to 40% by mass of polyoxyethylene glyceryl fatty acid ester, and (5) 15 to 88% by mass of water, wherein the weight ratio between the silicone oil (1) and the volatile hydrocarbon oil (2) ((1)/(2)) falls within the range of 0.3 to 3 and the composition has a viscosity of 100 to 500 mPas at 25° C.

### CROSS REFERENCE TO RELATED APPLICATIONS

This application relates to and claims priority from PCT/JP2009/005355 filed Oct. 14, 2009, the entire contents of which are incorporated herein by reference, which in turn claims priority from JP 2008-265965 filed Oct. 15, 2008 and JP 2009-234521 filed Oct. 8, 2009 (now granted and awaiting issue).

### FIGURE SELECTED FOR PUBLICATION

No figures

### BACKGROUND OF THE INVENTION

1. Field of the Invention

The present invention relates to a composition for external use on the skin, which contains tranexamic acids and has such a use feeling that a dewy feel and good absorption are achieved without a sticky or squeaky feel. More specifically, the present invention relates to a liquid composition for external use on the skin, which contains tranexamic acids and xanthan gum and has a viscosity adjusted to a specific value or less.

2. Description of the Related Art

Tranexamic acids, which have anti-plasmin activity, are added as an active ingredient for skin roughness amelioration, skin whitening etc. to various cosmetics. It was also found that a combination of tranexamic acids and another substance produced a synergistic effect or a new effect. For example, Patent Document 1 discloses a preparation for external use on the skin, which contains at least two ingredients selected from tranexamic acid, arbutin, trimethylglycine, and vitamin E, and also discloses that the skin external preparation can prevent skin dullness caused by various factors.

Patent Document 2 discloses that a skin cosmetic preparation containing tranexamic acid and the sap of a betulaceous plant such as white birch has a high anti-inflammatory effect. Patent Documents 3 and 4 disclose that the addition of tranexamic acid as a neutralizer or an emulsion stabilizer to a composition containing a carboxyvinyl polymer or an alkyl-modified carboxyvinyl polymer makes it possible to obtain a gel composition that has a high moisturizing effect and is well absorbed into the skin or to obtain an emulsion composition that is stable for a long term without addition of an additional surfactant.

Meanwhile, compositions for external use on the skin are required not to cause a sticky or squeaky feel during application to the skin. Patent Document 5 discloses that a preparation for external use on the skin containing a whitening ingredient such as tranexamic acid is produced using a thickener obtained by a process including dissolving a hydrophilic compound with gelation ability in water or an aqueous ingredient, cooling the solution to form a gel, and then crushing the gel into a predetermined particle size and that in that case, the whitening ingredient can be added at a high concentration without a reduction in viscosity or without a sticky or squeaky feel, which would otherwise be caused by a conventional thickener (such as a polysaccharide, a hydrophilic synthetic polymer, or a clay mineral). Patent Document 6 discloses that a combination of erythritol, tranexamic acid and a skin coating material is added to reduce the sticky feel caused by the addition of glycerin, a typical moisturizing material, but it neither discloses nor suggests a study on the sticky or squeaky feel caused by tranexamic acid.

### PRIOR ART DOCUMENTS

### Patent Documents

Patent Document 1: Japanese Patent Application Laid Open (JP-A) No. 10-265323

Patent Document 2: JP-A No. 9-291012

Patent Document 3: JP-A No. 9-124878

Patent Document 4: JP-A No. 9-263510

Patent Document 5: JP-A No. 2001-342125

Patent Document 6: JP-A No. 7-215839

### ASPECTS AND SUMMARY OF THE INVENTION

### Problems to be Solved by the Invention

When tranexamic acids are added to a low-viscosity liquid composition such as a lotion, there is a problem in which a sticky or squeaky feel is produced during application of the composition to the skin. This phenomenon becomes particularly significant when tranexamic acids are added in an amount of more than about 0.5% by mass. In the conventional art, it is recognized that high-viscosity compositions have a sticky or squeaky feel due to a surfactant or a thickener such as a polysaccharide or a clay mineral, but no study has been made yet regarding the sticky or squeaky feel of low-viscosity compositions due to the inclusion of tranexamic acids.

It is therefore an object of the present invention to provide a liquid composition for external use on the skin, which contains tranexamic acids, prevents a sticky or squeaky feel caused by the presence of tranexamic acids, and has such a use feeling that a dewy feel and good absorption are achieved.

### Means for Solving the Problems

As a result of studies to solve the above problems, the inventors have accomplished the present invention based on the finding that when a specific amount of xanthan gum is added to a composition containing tranexamic acids and being substantially free of thickening ingredients other than xanthan gum or when the composition contains another thickening ingredient in addition to xanthan gum under specific conditions and when the viscosity of the entire composition is adjusted to a specific value or less, a liquid composition for external use on the skin can be obtained, which prevents a sticky or squeaky feel caused by tranexamic acids, and has high stability and such a use feeling that a dewy feel and good absorption are achieved.

Thus, the present invention provides a liquid composition for external use on the skin, containing:

(A) at least one selected from tranexamic acid and derivatives thereof;

(B) 0.01 to 0.5% by mass of xanthan gum; and

(C) glycerin, and having a viscosity of 500 mPa·s or less at 30° C., wherein

the composition is substantially free of a thickening ingredient other than xanthan gum or contains a thickening ingredient other than xanthan gum under specific conditions.

### Effects of the Invention

Although the liquid composition of the present invention for external use on the skin contains tranexamic acids and glycerin, the specified amount of xanthan gum prevents a sticky or squeaky feel caused thereby, and the composition also has a dewy sensation in use and is well absorbed into the skin.

The above, and other aspects, features and advantages of the present invention will become apparent from the following description read in conjunction with the accompanying drawings, in which like reference numerals designate the same elements.

### BRIEF DESCRIPTION OF THE DRAWINGS

None

### DETAILED DESCRIPTION OF THE PREFERRED EMBODIMENTS

Reference will now be made in detail to embodiments of the invention that are illustrated in the discussion herein.

### MODES FOR CARRYING OUT THE INVENTION

The composition of the present invention contains at least one (ingredient A) selected from tranexamic acids. Tranexamic acid (trans-4-aminomethylcyclohexane-1-carboxylic acid) and derivatives thereof are generally used as an anti-plasmin agent and known to be an ingredient with a high level of safety for skin external preparations including cosmetics.

Tranexamic acids used in the composition of the present invention is not restricted and may be one conventionally used in cosmetics or the like, and include, besides tranexamic acid itself, for example, derivatives thereof including salts of tranexamic acid (such as metal salts such as magnesium, calcium, sodium, and potassium salts, a phosphate, a hydrochloride, a hydrobromate, and a sulfate), esters of tranexamic acid (such as vitamin esters such as a vitamin A acid ester, a vitamin A ester, a vitamin E ester, a vitamin C ester, and a vitamin D ester, phenyl esters, hydroquinone esters, and gentisic acid esters), and amides of tranexamic acid (such as a methyl amide or a salt thereof), and a dimer of tranexamic acid.

The content of tranexamic acids in the composition of the present invention is not restricted. When the content is 0.6% by mass or more, the composition can have not only a high skin-roughness-ameliorating or skin-whitening effect based on tranexamic acids but also a significant advantageous effect according to the present invention. Therefore, the composition of the invention preferably contains 0.6 to 5.0% by mass, more preferably 0.6 to 3.0% by mass of tranexamic acids.

In addition to tranexamic acids, the composition of the present invention contains 0.01 to 0.5% by mass of xanthan gum (ingredient B). Xanthan gum, one of known acidic polysaccharides, is natural gum which can be obtained by fermentation of a carbohydrate such as glucose with Xanthomonas bacteria. Also in the field of cosmetics, it is conventionally used as a water-soluble thickening ingredient, a moisturizing ingredient, or the like.

Xanthan gum contained in the composition of the present invention is not restricted and may be one conventionally used in cosmetics or the like. For example, a commercially available xanthan gum product such as KELTROL (trade name) manufactured by CPKelco, SAN-ACE (trade name) manufactured by San-Ei Gen F.F.I., Inc., or NOMUCOAT ZZ (trade name) manufactured by The Nisshin OilliO Group, Ltd. may be used without modification.

The content of xanthan gum in the composition of the present invention is from 0.01 to 0.5% by mass, preferably from 0.03 to 0.3% by mass. If the content is less than 0.01% by mass, a sticky or squeaky feel caused by tranexamic acid cannot be reduced, and if the content is more than 0.5% by mass, xanthan gum may cause a slimy or sticky feel.

The composition of the present invention further contains glycerin (ingredient C) as a moisturizing ingredient. The addition of glycerin makes it possible to moisturize the skin and to further increase the skin-roughness-ameliorating effect.

The content of glycerin in the composition of the present invention is generally, but not limited to, from 0.1 to 20% by mass, preferably from 1 to 10% by mass.

The composition of the present invention also essentially has a viscosity of 500 mPa's or less. If the viscosity is more than 500 mPa·s, the composition cannot be well absorbed into the skin and may be reduced in luxury characteristics such as a penetrating feel. The lower limit of the viscosity may be typically, but not limited to, 10 mPa·s or more.

The viscosity is determined using a VDA viscometer (DIGITAL VISMETRON VDA, Shibaura Systems Co., LTD.) and Rotor No. 1 or 2 under the conditions of a number of revolutions of 12 rmp and 1 minute.

For example, when only xanthan gum is contained as a thickening ingredient in the composition of the present invention, a viscosity of 500 mPa·s or less can be achieved by controlling the content of xanthan gum within the specified range according to the present invention (see Examples 1 to 4 below). Therefore, the composition of the present invention encompasses a composition substantially free of any other thickening ingredient than xanthan gum.

As used herein, the term “thickening ingredient” is intended to include not only a water-soluble thickening ingredient but also an oil-soluble (water-insoluble) thickening ingredient. In the composition, such an oil-soluble thickening ingredient should be present in the form of a combination with a suitable oil so that the thickening effect can be produced. On the other hand, the term “substantially free of” means that the composition does not contain a thickening ingredient in such a form as to produce the above thickening effect.

However, the addition of any other thickening ingredient to the composition of the present invention is not always excluded, and another thickening ingredient (ingredient D) may also be added under specific conditions in addition to the specified amount of xanthan gum, as long as the requirement that the viscosity of the entire the composition be 500 mPa·s or less is satisfied.

Other thickening ingredients that may be added to the composition of the present invention are not restricted and may be those conventionally used in cosmetics or the like, examples of which include water-soluble thickening ingredients such as hydroxymethylcellulose, hydroxyethylcellulose, hydroxypropylcellulose, carboxyvinyl polymers, alkyl-modified carboxyvinyl polymers, polyacrylamide, sodium alginate, propylene glycol alginate, agar, sodium polyacrylate, succinoglucan, dextran, mannan, marmelo, algae colloid, pectin, gellan gum, carrageenan, hyaluronic acid, polyvinyl alcohol, high polymerization degree polyethylene glycol, bentonite, magnesium aluminum silicate, Laponite, hectorite, and silicic anhydride, and oil-soluble thickening ingredients in combination with a suitable oil.

As used herein, the term “specific conditions” means that when a carboxyvinyl polymer is contained as another thickening ingredient, its content is 0.1% by mass or less based on the total weight of the composition and 500% by mass (five times) or less based on the weight of xanthan gum. On the other hand, when any other thickening ingredient than a carboxyvinyl polymer is added, its content is not restricted, as long as the requirement that the viscosity of the entire composition be 500 mP·s or less.

When the conditions are satisfied, the dewy feel or good absorption into the skin can be further improved without a sticky or squeaky feel even through another thickening ingredient is added in addition to xanthan gum. If a carboxyvinyl polymer is added at a concentration of more than 0.1% by mass based on the weight of the entire composition or added at a concentration of 0.1% by mass or less but in an amount more than five times the weight of xanthan gum, rather the carboxyvinyl polymer may cause a squeaky or sticky feel.

The composition of the present invention preferably further contains a hydrophilic amphipathic substance (ingredient E). The addition of the hydrophilic amphipathic substance makes it possible to further improve the good absorption into the skin during the application of the composition. When a liquid oil (ingredient F) is added, the hydrophilic amphipathic substance can also act as a solubilizing or emulsifying agent.

Any hydrophilic amphipathic substance conventionally used in cosmetics or the like may be added to the composition of the present invention. Examples of the hydrophilic amphipathic substance include, but are not limited to, polyoxyethylene fatty acid ether, polyoxyethylene fatty acid ester, polyoxyethylene alkyl ether, polyoxyethylene polyoxypropylene alkyl ether, polyoxyethylene allyl phenyl ether, polyoxyethylene cholesteryl ether, polyoxyethylene phytosterol ethers, polyoxyethylene polyoxypropylene phytosterol ether, polyoxyethylene hydrogenated castor oil, polyoxyethylene sorbitan fatty acid ester, polyoxyethylene sorbit fatty acid ester, polyoxyethylene fatty acid glycerin, polyglyceryl fatty acid ester, sucrose fatty acid ester, fatty acid soap, N-acylglutamic acid salt, acyltaurine salt, acylalkyltaurine salt, higher alkyl sulfate, alkyl ether sulfate, N-acylsarcosinate, higher fatty acid amide sulfonate, phosphate, sulfosuccinate, alkyl benzene sulfonate, and polyoxyethylene methyl ether dimethicone.

In particular, at least one selected from polyoxyethylene polyoxypropylene alkyl ether, polyoxyethylene hydrogenated castor oil, and polyoxyethylene phytosterol ether is preferably used.

The content of the hydrophilic amphipathic substance in the composition of the present invention is preferably, but not limited to, from 0.01 to 1.0% by mass, more preferably from 0.05 to 0.3% by mass. If the content is less than 0.01% by mass, it may be insufficient to improve the good absorption into the skin. If the content is more than 1.0% by mass, it may rather cause a sticky feel.

The composition of the present invention preferably further contains an oil (ingredient F) that is liquid at room temperature. The addition of the oil makes it possible to further improve the dewy feel or the good absorption into the skin during the application of the composition.

The liquid oil that may be added to the composition of the present invention is not restricted and may be one conventionally used in cosmetics or the like. For example, liquid oils conventionally used in cosmetics or the like, such as oils, fats, fatty acids, ester oils, hydrocarbon oils, higher alcohols, and silicone oils may be used. Specific examples thereof include those described below.

Examples of oils and fats include linseed oil, tsubaki oil, macadamia nut oil, corn oil, olive oil, avocado oil, sasanqua oil, castor oil, safflower oil, apricot seed oil, cinnamon oil, jojoba oil, grape oil, sunflower oil, almond oil, rapeseed oil, sesame oil, wheat germ oil, rice germ oil, rice bran oil, cotton seed oil, soybean oil, peanut oil, tea seed oil, and evening primrose oil.

Examples of fatty acids include heptanoic acid, octanoic acid, nonanoic acid, decanoic acid, pentadecanoic acid, heptadecanoic acid, nonadecanoic acid, isostearic acid, oleic acid, linoleic acid, linolenic acid, and arachidonic acid.

Examples of ester oils include pentaerythritol tetraoctanoate, cetyl octanoate, hexyl laurate, isopropyl myristate, octyldodecyl myristate, octyl palmitate, isopropyl isostearate, octyl isopalmitate, isodecyl oleate, and cetyl ethylhexanoate.

Examples of hydrocarbon oils include liquid paraffin, squalane, squalene, paraffin, isoparaffin, octane, decane, dodecane, isododecane, hexadecane, and isohexadecane.

Examples of higher alcohols include octyl alcohol, isostearyl alcohol, and oleyl alcohol.

Examples of silicone oils include chain silicones such as dimethylpolysiloxane, methylphenylpolysiloxane, and methylhydrogenpolysiloxane; and cyclic silicones such as octamethylcyclotetrasiloxane, decamethylcyclopentasiloxane, and dodecamethylcyclohexasiloxane.

The liquid oil is preferably added in combination with the hydrophilic amphipathic substance and/or a polymer having emulsifying ability or the like. When these ingredients coexist, the stability of the solubilized or emulsified liquid oil is improved. When emulsification is performed using the hydrophilic amphipathic substance, the oil droplets should have a small average emulsified particle size (for example, 300 nm or less), so that good stability and a good use feeling with a further dewy feel can be achieved.

The content of the liquid oil in the composition of the present invention is preferably, but not limited to, from 0.01 to 5.0% by mass, more preferably from 0.05 to 2.0% by mass. If the content is less than 0.01% by mass, it may be insufficient to increase the dewy feel. If the content is more than 5.0% by mass, a relatively large amount of the hydrophilic amphipathic substance and/or the polymer having emulsifying ability or the like should be added to stabilize the composition, which may cause a sticky feel.

As a non-limiting example, the composition of the present invention is typically suitable for use as a liquid cosmetic preparation such as a lotion (including a clear, translucent or cloudy lotion), an essence (a beauty essence), a liquid impregnant for sheet masks, or a double layer-type beauty essence. In addition, the composition, which contains tranexamic acid or the like, is particularly effective as a cosmetic preparation for ameliorating skin roughness or skin whitening.

Besides the ingredients described above, the composition of the present invention may further contain other ingredients generally used in cosmetics or the like.

Examples of other ingredients include, but are not limited to, moisturizing agents (other than glycerin), alcohols, lipophilic amphipathic substances, semi-solid oils, solid oils, drugs, buffers, perfumes, preservatives, colorants, sequestering agents, antioxidants, ultraviolet absorbing agents, and powders.

### EXAMPLES

Hereinafter, the present invention is described in more detail and specifically with reference to some examples, which however are not intended to limit the scope of the invention. Unless otherwise stated, the content is expressed in % by mass in all of the examples, the comparative examples, and the formulation examples below.

### Examples 1 to 7 and Comparative Examples 1 to 9

Compositions were prepared, which were composed as shown in Tables 1 to 4 below. These compositions were measured for viscosity at 30° C., and the compositions containing liquid oil in the form of an emulsion were measured for the average emulsified particle size of the dispersed oil droplets. The results are shown in Tables 1 to 4.

Next, these compositions were subjected to an actual use test by a panel of 20 experts. The test items were “dewy feel,” “absorption into the skin,” “the absence of stickiness during drying,” and “the absence of squeaky feel during drying.” Each panel expert evaluated each test item according to the evaluation score criteria below, and categorized the total evaluation stores into four ranks. The ranking results are also shown in Tables 1 to 4.

### Evaluation Score Criteria

5: excellent
4: good
3: fair
2: poor
1: very poor

### Evaluation Ranking

A: The total score is 80 or more.
B: The total score is from 60 to less than 80.
C: The total score is from 40 to less than 60.
D: The total score is less than 40.

TABLE 1

| | Example 1 | Example 2 | Example 3 | Example 4 |
|---|---|---|---|---|
| Water | Balance | Balance | Balance | Balance |
| Tranexamic acid | 1 | 2 | 3 | — |
| Tranexamic acid | — | — | — | 5 |
| methyl amide | | | | |
| hydrochloride | | | | |
| Xanthan gum | 0.03 | 0.1 | 0.3 | 0.5 |
| Polyoxyethylene | 0.1 | — | — | — |
| polyoxypropylene decyl | | | | |
| tetradecyl ether | | | | |
| Polyoxyethylene | — | 0.2 | — | — |
| hydrogenated castor oil | | | | |
| Polyoxyethylene glyceryl | — | — | 0.5 | — |
| isostearate | | | | |
| Polyoxyethylene | — | — | 0.2 | 0.2 |
| phytosterol | | | | |
| Glycerin | 5 | 1 | 8 | 3 |
| Dipropylene glycol | 3 | 5 | 4 | 1 |
| Polyglyceryl diisostearate | — | 0.2 | 0.5 | — |
| Glyceryl | — | 0.1 | 1 | — |
| triethylhexanoate | | | | |
| Ethyl alcohol | 3 | 6 | 10 | — |
| Methylparaben | q.s. | q.s. | q.s. | q.s. |
| Edetate | q.s. | q.s. | q.s. | q.s. |
| Citric acid | q.s. | q.s. | q.s. | q.s. |
| Sodium citrate | q.s. | q.s. | q.s. | q.s. |
| Perfume | q.s. | q.s. | q.s. | q.s. |
| Total | 100 | 100 | 100 | 100 |
| Viscosity (mPa · s) | 25 | 200 | 360 | 480 |
| Average emulsified | — | 180 | 240 | — |
| particle size (nm) | | | | |
| Dewy feel | B | A | A | B |
| Absorption into the skin | A | A | A | B |
| Absence of stickiness | A | A | A | B |
| during drying | | | | |
| Absence of squeaky | B | A | A | A |
| feel during drying | | | | |

TABLE 2

| | Example 5 | Example 6 | Example 7 |
|---|---|---|---|
| Water | Balance | Balance | Balance |
| Tranexamic acid | 2 | — | — |
| Tranexamic acid methyl amide | — | 3 | 4 |
| hydrochloride | | | |
| Xanthan gum | 0.1 | 0.02 | 0.02 |
| Hydroxyethylcellulose | 0.2 | — | — |
| Carboxyvinyl polymer | — | 0.07 | 0.1 |
| Polyoxyethylene polyoxypropylene | — | 0.5 | 0.2 |
| decyl tetradecyl ether | | | |
| Polyoxyethylene hydrogenated castor | 0.1 | — | — |
| oil | | | |
| Glycerin | 10 | 4 | 1 |
| Dipropylene glycol | — | 10 | 8 |
| Ethyl alcohol | 10 | 5 | 3 |
| Methylparaben | q.s. | q.s. | q.s. |
| Edetate | q.s. | q.s. | q.s. |
| Citric acid | q.s. | q.s. | q.s. |
| Sodium citrate | q.s. | q.s. | q.s. |
| Caustic potash | — | q.s. | q.s. |
| Perfume | q.s. | q.s. | q.s. |
| Total | 100 | 100 | 100 |
| Viscosity (mPa · s) | 300 | 180 | 250 |
| Dewy feel | A | A | A |
| Absorption into the skin | B | A | A |
| Absence of stickiness during drying | A | B | B |
| Absence of squeaky feel during | A | B | B |
| drying | | | |

TABLE 3

| | Com- | Com- | Com- | Com- |
|---|---|---|---|---|
| | parative | parative | parative | parative |
| | Example 1 | Example 2 | Example 3 | Example 4 |
| Water | Balance | Balance | Balance | Balance |
| Tranexamic acid | 1 | 2 | 0.6 | 3 |
| Xanthan gum | — | 0.7 | 0.3 | — |
| Hydroxyethylcellulose | — | — | 0.2 | — |
| Polyoxyethylene | 0.1 | — | — | — |
| polyoxypropylene decyl | | | | |
| tetradecyl ether | | | | |
| Polyoxyethylene | — | — | 0.1 | — |
| hydrogenated castor oil | | | | |
| Polyoxyethylene glyceryl | — | — | — | 0.5 |
| isostearate | | | | |
| Polyoxyethylene | — | 0.2 | — | 0.1 |
| phytosterol | | | | |
| Glycerin | 5 | 1 | 10 | 8 |
| Dipropylene glycol | 3 | 5 | — | 4 |
| Polyglyceryl diisostearate | — | — | — | 0.5 |
| Glyceryl | — | — | — | 1 |
| triethylhexanoate | | | | |
| Ethyl alcohol | 3 | 6 | 10 | 8 |
| Methylparaben | q.s. | q.s. | q.s. | q.s. |
| Edetate | q.s. | q.s. | q.s. | q.s. |
| Citric acid | q.s. | q.s. | q.s. | q.s. |
| Sodium citrate | q.s. | q.s. | q.s. | q.s. |
| Perfume | q.s. | q.s. | q.s. | q.s. |
| Total | 100 | 100 | 100 | 100 |
| Viscosity (mPa · s) | — | 830 | 600 | — |
| Average emulsified | — | — | — | 240 |
| particle size (nm) | | | | |
| Dewy feel | D | A | C | C |
| Absorption into the skin | A | D | C | A |
| Absence of stickiness | C | D | B | D |
| during drying | | | | |
| Absence of squeaky feel | D | A | A | D |
| during drying | | | | |

TABLE 4

| | | | Com- | | Com- |
|---|---|---|---|---|---|
| | Com- | Com- | parative | Com- | parative |
| | parative | parative | Ex- | parative | Ex- |
| | Example 5 | Example 6 | ample 7 | Example 8 | ample 9 |
| Water | Balance | Balance | Balance | Balance | Balance |
| Tranexamic acid | — | 2 | 1 | — | 5 |
| Tranexamic acid | 2 | — | — | 3 | — |
| methyl amide | | | | | |
| hydrochloride | | | | | |
| Xanthan gum | — | — | 0.2 | 0.01 | — |
| Veegum | — | — | — | — | 2 |
| Hydrogenated | — | — | — | — | 1 |
| olive oil | | | | | |
| Carboxyvinyl | 0.1 | — | 0.12 | 0.06 | — |
| polymer | | | | | |
| Polyacrylamide | — | 0.1 | — | — | — |
| Polyoxyethylene | — | 0.2 | — | 0.1 | — |
| polyoxypropylene | | | | | |
| decyl tetradecyl | | | | | |
| ether | | | | | |
| Polyoxyethylene | — | — | 0.1 | — | — |
| hydrogenated | | | | | |
| castor oil | | | | | |
| Polyoxyethylene | 0.2 | — | — | — | 2.0 |
| glyceryl | | | | | |
| isostearate | | | | | |
| Polyoxyethylene | — | — | — | 0.1 | — |
| phytosterol | | | | | |
| Glycerin | 3 | 2 | 4 | 7 | 10 |
| Dipropylene | 1 | 7 | 3 | 5 | 2 |
| glycol | | | | | |
| Ethyl alcohol | 2 | 5 | 1 | 8 | — |
| Methylparaben | q.s. | q.s. | q.s. | q.s. | q.s. |
| Edetate | q.s. | q.s. | q.s. | q.s. | q.s. |
| Citric acid | q.s. | q.s. | q.s. | q.s. | q.s. |
| Sodium citrate | q.s. | q.s. | q.s. | q.s. | q.s. |
| Caustic potash | q.s. | — | q.s. | q.s. | q.s. |
| Perfume | q.s. | q.s. | q.s. | q.s. | q.s. |
| Total | 100 | 100 | 100 | 100 | 100 |
| Viscosity | 200 | 300 | 450 | 80 | — |
| (mPa · s) | | | | | |
| Dewy feel | A | A | A | B | C |
| Absorption into | C | C | C | A | C |
| the skin | | | | | |
| Absence of | C | C | C | C | D |
| stickiness during | | | | | |
| drying | | | | | |
| Absence of | D | D | C | C | B |
| squeaky feel | | | | | |
| during drying | | | | | |

Tranexamic acids and xanthan gum, which are essential ingredients of the composition of the present invention, are conventionally used in cosmetics. Therefore, for example, Patent Documents 1, 2 and 5 disclose compositions containing both materials. However, the cream and the gel disclosed in Patent Document 1 (Table 3) and Patent Document 5 (Example 12) are high-viscosity compositions. Patent Document 2 (Example 11) discloses an example where an essence contains both materials, in which, however, the content of tranexamic acid is 0.5% by mass, and therefore there is no need to take into account an unfavorable use feeling caused by tranexamic acid, such as a sticky or squeaky feel. In addition, the essence also contains 0.2% by mass of hydroxyethylcellulose sodium in addition to 0.3% by mass of xanthan gum, and therefore, in terms of absorption into the skin or dewy feel, the essence is considered to be substantially equivalent to a composition that is the same as that of Comparative Example 3, except that water is partially replaced by white birch sap, which is different from the composition within the scope of the invention. Patent Documents 3 and 4 disclose compositions containing tranexamic acid and a carboxyvinyl polymer or an alkyl-modified carboxyvinyl polymer, but the compositions, which do not contain xanthan gum, are considered to be as inferior as Comparative Example 5 with respect to sticky or squeaky feel and absorption into the skin. Patent Document 6 (Table 3) discloses test results showing that the sticky feel of a composition containing glycerin and tranexamic acid at high concentrations is reduced by the replacement of glycerin with erythritol. In contrast, the present invention has been accomplished based on the new finding that the addition of a specific amount of xanthan gum makes it possible to obtain a glycerin and tranexamic acid-containing skin external preparation that has no sticky or squeaky feel even without erythritol.

### Formulation Example 1

### Lotion

| Ingredients | Content (% by mass) |
|---|---|
| Tranexamic acid | 1.0 |
| Potassium 4-methoxysalicylate | 2.0 |
| Glycerin | 8.0 |
| Butylene glycol | 4.0 |
| Hyaluronic acid | 0.01 |
| Xanthan gum | 0.05 |
| PEG/PPG-17/4 dimethyl ether | 3.0 |
| PEG-13 decyl tetradecyl ether-24 | 0.2 |
| PEG-10 methyl ether dimethicone | 0.5 |
| Citric acid | q.s. |
| Sodium citrate | q.s. |
| Sodium metaphosphate | q.s. |
| Sodium pyrosulfite | q.s. |
| Phenoxyethanol | q.s. |
| Perfume | q.s. |
| Ion-exchanged water | balance |

### Preparation Method:

According to a conventional method, the water-soluble ingredients were sequentially dissolved in ion-exchanged water to prepare an aqueous phase. The water-insoluble ingredients were mixed with the amphipatic ingredient, and then the mixture was added to the aqueous phase so that a lotion was obtained.

### Formulation Example 2

### Cloudy Lotion

| Ingredients | Content (% by mass) |
|---|---|
| Tranexamic acid | 3.0 |
| Ethyl vitamin C | 1.5 |
| Dipotassium glycyrrhizinate | 0.05 |
| Glycerin | 1.0 |
| Dipropylene glycol | 4.0 |
| Polyethylene glycol 20000 | 1.0 |
| Ethanol | 10.0 |
| Xanthan gum | 0.1 |
| POE hydrogenated castor oil | 0.3 |
| Polyglyceryl diisostearate | 0.4 |
| Glyceryl triethylhexanoate | 0.2 |
| Squalane | 0.05 |
| Citric acid | q.s. |
| Sodium citrate | q.s. |
| Sodium metaphosphate | q.s. |
| Sodium pyrosulfite | q.s. |
| Methylparaben | q.s. |
| Perfume | q.s. |
| Dye | q.s. |
| Ion-exchanged water | balance |

### Preparation Method:

According to a conventional method, the water-soluble ingredients were sequentially dissolved in ion-exchanged water to prepare an aqueous phase. The water-insoluble ingredients were heated and mixed with the amphipatic ingredient, and then the mixture was gradually added to the aqueous phase so that a cloudy lotion was obtained.

### Formulation Example 3

### Essence

| Ingredients | Content (% by mass) |
|---|---|
| Tranexamic acid methyl amide hydrochloride | 1.0 |
| Ascorbic acid glucoside | 2.0 |
| Dipotassium glycyrrhizinate | 0.05 |
| Glycerin | 10.0 |
| Propylene glycol | 5.0 |
| Maltitol | 3.0 |
| Xylitol | 2.0 |
| Acetylated hyaluronic acid | 0.03 |
| Xanthan gum | 0.2 |
| Hydroxyethylcellulose | 0.05 |
| Acrylic acid/alkyl (C10-30) acrylate copolymer | 0.05 |
| Methylphenylpolysiloxane | 2.0 |
| Cetyl octanoate | 1.0 |
| Isostearyl alcohol | 1.0 |
| Citric acid | q.s. |
| Sodium citrate | q.s. |
| Edetate | q.s. |
| Phenoxyethanol | q.s. |
| Perfume | q.s. |
| Ion-exchanged water | balance |

### Preparation Method:

According to a conventional method, the water-soluble ingredients were sequentially dissolved in ion-exchanged water to prepare an aqueous phase. The water-insoluble ingredients were mixed together, and then the mixture was gradually added to the aqueous phase and homogenized so that an essence was obtained.

### Formulation Example 4

### Essence

| Ingredients | Content (% by mass) |
|---|---|
| Tranexamic acid methyl amide hydrochloride | 2.5 |
| Potassium 4-methoxysalicylate | 0.5 |
| Nicotinamide | 4.0 |
| Tocopherol acetate | 0.05 |
| Glycerin | 1.0 |
| PEG/PPG-17/4 dimethyl ether | 5.0 |
| Polyethylene glycol 1500 | 1.0 |
| Erythritol | 1.0 |
| Trimethylglycine | 2.5 |
| Ethanol | 3.0 |
| Xanthan gum | 0.08 |
| Sodium polyacrylate | 0.03 |
| Sodium alginate | 0.02 |
| POE phytosterol | 0.2 |
| Polyglyceryl diisostearate | 0.1 |
| Glyceryl trihethylhexanoate | 0.1 |
| Di(phytosteryl/octyldodecyl) lauroylglutamate | 0.1 |
| Citric acid | q.s. |
| Sodium citrate | q.s. |
| Sodium metaphosphate | q.s. |
| Salicylic acid | q.s. |
| Perfume | q.s. |
| Dye | q.s. |
| Ion-exchanged water | balance |

### Preparation Method:

According to a conventional method, the water-soluble ingredients were sequentially dissolved in ion-exchanged water to prepare an aqueous phase. The water-insoluble ingredients were heated and mixed with the amphipatic ingredient, and then the mixture was gradually added to the aqueous phase so that an essence was obtained.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

1. A liquid composition for external use on skin, comprising:
(A) 1.0 to 5.0% by mass of at least one selected from tranexamic acid, salts of tranexamic acid and amides of tranexamic acid;
(B) 0.01 to 0.3% by mass of xanthan gum;
(C) glycerin, and
(E) at least one hydrophilic amphipathic substance selected from the group consisting of polyoxyethylene fatty acid ether, polyoxyethylene fatty acid ester, polyoxyethylene alkyl ether, polyoxyethylene polyoxypropylene alkyl ether, polyoxyethylene alkyl phenyl ether, polyoxyethylene cholesteryl ether, polyoxyethylene phytosterol ethers, polyoxyethylene polyoxypropylene phytosterol ether, polyoxyethylene hydrogenated castor oil, polyoxyethylene sorbitan fatty acid ester, polyoxyethylene sorbit fatty acid ester, and polyoxyethylene fatty acid glycerin; and
having a viscosity of 500 mPa·s or less at 30° C., wherein
the composition is substantially free of a thickening ingredient other than the xanthan gum.

2. A liquid composition for external use on skin, comprising:
(A) 1.0 to 5.0% by mass of at least one selected from tranexamic acid, salts of tranexamic acid and amides of tranexamic acid;
(B) 0.01 to 0.3% by mass of xanthan gum;
(C) glycerin;
(D) a thickening ingredient other than xanthan gum, and
(E) at least one hydrophilic amphipathic substance selected from the group consisting of polyoxyethylene fatty acid ether, polyoxyethylene fatty acid ester, polyoxyethylene alkyl ether, polyoxyethylene polyoxypropylene alkyl ether, polyoxyethylene alkyl phenyl ether, polyoxyethylene cholesteryl ether, polyoxyethylene phytosterol ethers, polyoxyethylene polyoxypropylene phytosterol ether, polyoxyethylene hydrogenated castor oil, polyoxyethylene sorbitan fatty acid ester, polyoxyethylene sorbit fatty acid ester, and polyoxyethylene fatty acid glycerin; and
having a viscosity of 500 mPa·s or less at 30° C., wherein
when the thickening ingredient other than xanthan gum is a carboxyvinyl polymer, the content of the carboxyvinyl polymer is 0.1% by mass or less based on the total weight of the composition and 500% by mass or less based on the weight of xanthan gum.

3. The composition according to claim 1 or 2, further comprising (F) a liquid oil.

4. The composition according to claim 1, wherein the composition is for ameliorating skin roughness or skin whitening.

5. (canceled)

6. (canceled)
